(19) United States
(12) Patent Application Publication
(10) Pub. No.: US 2024/0021429 A1
(43) Pub. Date: Jan. 18, 2024

(54) METHOD FOR MANUFACTURING SEMICONDUCTOR SUBSTRATE AND COMPOSITION

(71) Applicant: JSR CORPORATION, Tokyo (JP)

(72) Inventors: Ken MARUYAMA, Tokyo (JP); Takayoshi Abe, Tokyo (JP); Kazunori Sakai, Tokyo (JP)

(73) Assignee: JSR CORPORATION, Tokyo (JP)

(21) Appl. No.: 18/372,163

(22) Filed: Sep. 25, 2023

Related U.S. Application Data
(63) Continuation-in-part of application No. PCT/JP22/10895, filed on Mar. 11, 2022.

(30) Foreign Application Priority Data
Mar. 26, 2021 (JP) 2021-053250
Nov. 26, 2021 (JP) 2021-192296

(51) Int. Cl. H01L 21/027; H01L 21/311; G03F 7/16; G03F 7/004; G03F 7/20

(52) U.S. Cl. CPC H01L 21/0274; H01L 21/31116; G03F 7/167; G03F 7/0042; G03F 7/2004

(57) ABSTRACT

A method for manufacturing a semiconductor substrate includes forming a resist underlayer film directly or indirectly on a substrate by applying a composition for forming a resist underlayer film. A metal-containing resist film is formed on the resist underlayer film. The metal-containing resist film is exposed. An exposed portion of the exposed metal-containing resist film is dissolved with a developer to form a resist pattern.

### CROSS-REFERENCE TO RELATED APPLICATIONS

The present application is a continuation-in-part application of International Patent Application No. PCT/JP2022/010895 filed Mar. 11, 2022, which claims priority to Japanese Patent Application No. 2021-053250 filed Mar. 26, 2021, and to Japanese Patent Application No. 2021-192296 filed Nov. 26, 2021. The contents of these applications are incorporated herein by reference in their entirety.

### BACKGROUND OF THE DISCLOSURE

### Technical Field

The present disclosure relates to a method for manufacturing a semiconductor substrate and a composition.

### Background Art

In a general method for forming a pattern, which is used for microfabrication by lithography, a resist film formed from a radiation-sensitive composition for forming a resist film is exposed to an electromagnetic wave such as an ultraviolet ray, a far ultraviolet ray (e.g., an ArF excimer laser beam or a KrF excimer laser beam) or an extreme ultraviolet ray (EUV), or a charged corpuscular ray such as an electron beam to generate an acid in an exposed portion. Then, a chemical reaction using this acid as a catalyst causes a difference in dissolution rate with respect to the developer between the exposed portion and the unexposed portion, and a pattern is thereby formed on a substrate. The pattern formed can be used as a mask or the like in substrate processing. Such a method for forming a pattern is required to improve resist performance along with miniaturization of processing technology. In response to this requirement, the types, molecular structures, and so on of an organic polymer, an acid generator, and other components to be used in a radiation-sensitive composition for forming a resist film have been studied, and combinations thereof have also been studied in detail (see JP-A-2000-298347). It has also been studied to use a metal-containing compound instead of the organic polymer.

### SUMMARY

According to an aspect of the present disclosure, a method for manufacturing a semiconductor substrate includes forming a resist underlayer film directly or indirectly on a substrate by applying a composition for forming a resist underlayer film. A metal-containing resist film is formed on the resist underlayer film. The metal-containing resist film is exposed. An exposed portion of the exposed metal-containing resist film is dissolved with a developer to form a resist pattern.

According to another aspect of the present disclosure, a composition includes: at least one component selected from the group consisting of an acid generating component, an acid group-containing component, a photo-base generator, and a base-containing component; and a solvent. The composition is suitable for forming a resist underlayer film in a method for manufacturing a semiconductor substrate. The method includes forming a resist underlayer film directly or indirectly on a substrate by applying the composition. A metal-containing resist film is formed on the resist underlayer film. The metal-containing resist film is exposed. An exposed portion of the exposed metal-containing resist film is dissolved with a developer to form a resist pattern.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

In a resist pattern formed using the metal-containing compound described above, there may be a case where the resist pattern collapses or trailing of the pattern at the bottom of a resist film occurs.

The present invention relates to, in one embodiment, a method for manufacturing a semiconductor substrate, the method including:
applying a composition for forming a resist underlayer film directly or indirectly to a substrate;
forming a metal-containing resist film on a resist underlayer film formed by applying the composition for forming a resist underlayer film;
exposing the metal-containing resist film;
preparing a developer; and
dissolving an exposed portion of the exposed metal-containing resist film with the developer to form a resist pattern.

In another embodiment, the present invention relates to a composition for forming a resist underlayer film to be used for a method for manufacturing a semiconductor substrate, the method including:
applying a composition for forming a resist underlayer film directly or indirectly to a substrate;
forming a metal-containing resist film on a resist underlayer film formed by applying the composition for forming a resist underlayer film;
exposing the metal-containing resist film;
preparing a developer; and
dissolving an exposed portion of the exposed metal-containing resist film with the developer to form a resist pattern,
wherein the composition for forming a resist underlayer film includes:
at least one component selected from the group consisting of an acid generating component, an acid group-containing component, a photo-base generator, and a base-containing component; and
a solvent.

According to the method for manufacturing a semiconductor substrate, a composition for forming a resist underlayer film capable of forming a resist underlayer film superior in resist pattern rectangularity is used, so that a semiconductor substrate having a good pattern shape can be efficiently manufactured. When the composition for forming a resist underlayer film is used, a resist underlayer film having superior resist pattern rectangularity can be formed, so that a semiconductor substrate having a good pattern shape can be efficiently manufactured. Therefore, the method for manufacturing a semiconductor substrate and the composition for forming a resist underlayer film can be suitably used for manufacturing a semiconductor device which is expected to be further miniaturized in the future.

Hereinafter, a method for manufacturing a semiconductor substrate and a composition for forming a resist underlayer film according to each embodiment of the present invention will be described in detail.

### «Method for Manufacturing Semiconductor Substrate»

The method for manufacturing a semiconductor substrate includes applying a composition for forming a resist underlayer film directly or indirectly to a substrate (this step is hereinafter also referred to as “composition for forming a resist underlayer film application step”); forming a metal-containing resist film on a resist underlayer film formed by the composition for forming a resist underlayer film application step (this step is hereinafter also referred to as “metal-containing resist film formation step”); exposing the metal-containing resist film formed by the metal-containing resist film formation step (this step is hereinafter also referred to as “exposure step”); preparing a developer (this step is hereinafter also referred to as “developer preparation step”); and dissolving an exposed portion of the exposed metal-containing resist film with the developer to form a resist pattern (this step is hereinafter also referred to as “resist pattern formation step”). The developer preparation step may be performed at any stage before the resist pattern formation step.

Hereinafter, each step of the method for manufacturing a semiconductor substrate will be described.

### [Composition for Forming a Resist Underlayer Film Application Step]

In this step, a composition for forming a resist underlayer film is applied directly or indirectly to a substrate. The method of the application of the composition for forming a resist underlayer film is not particularly limited, and the application can be performed by an appropriate method such as spin coating, cast coating, or roll coating. As a result, a coating film is formed, and volatilization of the solvent in the composition for forming a resist underlayer film occurs, so that a resist underlayer film is formed. The composition for forming a resist underlayer film will be described later.

Next, the coating film formed by the application is heated. The formation of the resist underlayer film is promoted by the heating of the coating film. More specifically, volatilization or the like of the solvent in the composition for forming a resist underlayer film is promoted by the heating of the coating film.

The heating of the coating film may be performed either in the air atmosphere or in a nitrogen atmosphere. The lower limit of the heating temperature is preferably 100° C., more preferably 150° C., and still more preferably 200° C. The upper limit of the heating temperature is preferably 400° C., more preferably 350° C., and still more preferably 280° C. The lower limit of the heating time is preferably 15 seconds, and more preferably 30 seconds. The upper limit of the time is preferably 1,200 seconds, and more preferably 600 seconds.

The lower limit of the average thickness of the resist underlayer film to be formed is preferably 0.5 nm, more preferably 1 nm, and still more preferably 2 nm. The upper limit of the average thickness is preferably 50 nm, more preferably 20 nm, still more preferably 10 nm, and particularly preferably 7 nm. The average thickness is measured as described in Examples.

### [Metal-Containing Resist Film Formation Step]

In this step, a metal-containing resist film is formed on the resist underlayer film formed by the composition for forming a resist underlayer film application step.

The metal-containing resist film can be formed by depositing a metal compound on the resist underlayer film.

The deposition of the metal compound on the resist underlayer film may be performed by vapor deposition by chemical vapor deposition (CVD) or atomic layer deposition (ALD). The vapor deposition may be performed by plasma-enhanced (PE) CVD or plasma-enhanced (PE) ALD.

The deposition temperature by ALD may be 50° C. to 600° C. The deposition pressure by ALD may be 100 to 6000 mTorr. The flow rate of the metal compound by ALD may be 0.01 to 10 ccm and the gas flow rate (CO₂, CO, Ar, N₂) may be 100 to 10000 sccm. Plasma power by ALD may be 200 to 1000 W per 300 mm wafer station using radio-frequency plasma (e.g., 13.56 MHz, 27.1 MHz, or a frequency higher than 27.1 MHz).

Processing conditions suitable for vapor deposition by CVD include a deposition temperature of about 250° C. to 350° C. (for example, 350° C.), a reactor pressure of less than 6 Torr (for example, maintained at 1.5 to 2.5 Torr at 350° C.), a plasma power/bias of 200 W per 300 mm wafer station with a radio-frequency plasma (for example, 13.56 MHz or more), a metal compound flow rate of about 100 to 500 ccm, and a CO₂ flow rate of about 1000 to 2000 sccm.

Examples of the metal compound include trimethyltin chloride, dimethyltin dichloride, methyltin trichloride, tris(dimethylamino)methyltin(IV), and (dimethylamino)trimethyltin(IV).

The metal-containing resist film preferably includes an organotin oxide. Examples of the organotin oxide include oxides of organic metals such as a haloalkyl Sn, an alkoxyalkyl Sn, and an amidoalkyl Sn.

### [Exposure Step]

In this step, the metal-containing resist film formed by the metal-containing resist film formation step is exposed. This step causes a difference in solubility in the developer between an exposed portion and an unexposed portion in the metal-containing resist film. More specifically, the solubility of the exposed portion in the developer in the metal-containing resist film is increased.

The radiation to be used for the exposure can be appropriately selected according to the type of the metal-containing resist film to be used, and so on. Examples of the radiation include electromagnetic waves such as visible rays, ultraviolet rays, far ultraviolet rays, X-rays, and γ-rays and corpuscular rays such as electron beams, molecular beams, and ion beams. Among these, far ultraviolet rays are preferable, and a KrF excimer laser beam (wavelength: 248 nm), an ArF excimer laser beam (wavelength: 193 nm), an F₂ excimer laser beam (wavelength: 157 nm), a Kr₂ excimer laser beam (wavelength: 147 nm), an ArKr excimer laser beam (wavelength: 134 nm) or extreme ultraviolet rays (wavelength: 13.5 nm, etc., also referred to as “EUV”) are more preferred, and an ArF excimer laser beam or EUV is still more preferred. In addition, exposure conditions can be appropriately determined according to the type of the metal-containing resist film to be used, and so on.

The EUV exposure causes a dimerization reaction of the organotin oxide in the exposed portion of the metal-containing resist film. For example, CH₃Sn(SnO)₃, which is an organotin oxide, can yield Sn₂((SnO)₃)₂ by a dimerization reaction caused by the EUV exposure.

In this step, post exposure baking (hereinafter, also referred to as “PEB”) can be performed after the exposure in order to improve the resist film performance such as resolution, pattern profile, and developability. The PEB temperature and the PEB time may be appropriately determined according to the type of the material for forming the metal-containing resist film to be used, and so on. The lower limit of the PEB temperature is preferably 50° C., and more preferably 70° C. The upper limit of the PEB temperature is preferably 500° C., and more preferably 300° C. The lower limit of the PEB time is preferably 10 seconds, and more preferably 30 seconds. The upper limit of the PEB time is preferably 600 seconds, and more preferably 300 seconds.

### [Developer Preparation Step]

In this step, a developer is prepared. Examples of the developer include water, alcohol-based liquids, and ether-based liquids, and two or more kinds thereof may be used in combination.

Examples of the alcohol-based liquid include
monoalcohol-based liquids such as methanol, ethanol, n-propanol, iso-propanol, n-butanol, iso-butanol, sec-butanol, t-butanol, n-pentanol, iso-pentanol, sec-pentanol, t-pentanol, 2-methylpentanol, and 4-methyl-2-pentanol.

Examples of the ether-based liquid include
polyhydric alcohol partial ether-based solvents such as ethylene glycol monomethyl ether, ethylene glycol monoethyl ether, ethylene glycol dimethyl ether, and propylene glycol monoethyl ether, and polyhydric alcohol partial ether acetate-based liquids such as ethylene glycol monomethyl ether acetate, ethylene glycol monoethyl ether acetate, propylene glycol monomethyl ether acetate (PGMEA), and propylene glycol monoethyl ether acetate.

As the developer, water and alcohol-based liquids are preferable, and water, ethanol, or a combination thereof is more preferable.

In this step, heating may be performed. The lower limit of the heating temperature is preferably 20° C., and more preferably 30° C. The upper limit of the heating temperature is preferably 70° C., and more preferably 60° C.

### [Resist Pattern Formation Step]

In this step, the exposed portion of the exposed metal-containing resist film is dissolved with the developer to form a resist pattern. The dimerization reaction product of the organotin oxide in the metal-containing resist film is dissolved with the developer to develop the metal-containing resist film. Specifically, Sn₂((SnO)₃)₂ generated through the dimerization reaction by EUV exposure is dissolved in the developer, and the metal-containing resist film is thereby developed to form a resist pattern.

The temperature of the developer can be appropriately determined according to the type of the material for forming the metal-containing resist film to be used, and so on. The lower limit of the temperature of the developer is preferably 20° C., more preferably 30° C., and still more preferably 40° C. The upper limit of the temperature of the developer is preferably 70° C., and more preferably 60° C. The lower limit of the developing time is preferably 10 seconds, and more preferably 30 seconds. The upper limit of the developing time is preferably 600 seconds, and more preferably 300 seconds.

In this step, the exposed portion of the exposed metal-containing resist film may be dissolved with the developer, and then washing and/or drying may be performed.

### [Etching Step]

In this step, etching is performed using the resist pattern as a mask. The number of times of the etching may be once. Alternatively, etching may be performed a plurality of times, that is, etching may be sequentially performed using a pattern obtained by etching as a mask. Examples of an etching method include dry etching and wet etching. As a result of the etching, a semiconductor substrate having a prescribed pattern is obtained.

The dry etching can be performed using, for example, a publicly known dry etching apparatus. The etching gas used for dry etching can be appropriately selected according to the elemental composition of the silicon-containing film to be etched, and for example, fluorine-based gases such as CHF₃, CF₄, C₂F₆, C₃F₈, and SF₆, chlorine-based gases such as Cl₂ and BCl₃, oxygen-based gases such as O₂, O₃, and H₂O, reducing gases such as H₂, NH₃, CO, CO₂, CH₄, C₂H₂, C₂H₄, C₂H₆, C₃H₄, C₃H₆, C₃H₈, HF, HI, HBr, HCl, NO and BCl₃, and inert gases such as He, N₂ and Ar are used. These gases can also be used in admixture.

### «Composition for Forming a Resist Underlayer Film»

The composition for forming a resist underlayer film is used for a method for manufacturing a semiconductor substrate, the method including applying a composition for forming a resist underlayer film directly or indirectly to a substrate; forming a metal-containing resist film on a resist underlayer film formed by applying the composition for forming a resist underlayer film; exposing the metal-containing resist film; preparing a developer; and dissolving an exposed portion of the exposed metal-containing resist film with the developer to form a resist pattern. As details of respective steps, the steps of the method for manufacturing a semiconductor substrate can be suitably employed. The composition for forming a resist underlayer film includes at least one component selected from the group consisting of an acid generating component [A], an acid group-containing component [B], a photo-base generator [C1], and a base-containing component [C2], and a solvent [E].

(Acid generating component [A])

Examples of the acid generating component [A] include a thermal acid generator (hereinafter, also referred to as thermal acid generator [A1]), a thermally acid-generating polymer (hereinafter, also referred to as a thermally acid-generating polymer [A2]), and a photo-acid generator (hereinafter, also referred to as a photo-acid generator [A3]). The acid generating component [A] may be used singly or two or more kinds thereof may be used in combination.

### [Thermal Acid Generator [A1]]

The thermal acid generator [A1] is a component of a compound that generates, by an action of heat, a component having an acid group (hereinafter, also referred to as an “acid group (a)”) which is a sulfo group, a carboxy group, a phosphono group, a phosphoric acid group, a sulfuric acid group, a sulfonamide group, a sulfonylimide group, —CR^{F1}R^{F2}OH (R^{F1} is a fluorine atom or a fluorinated alkyl group; and R^{F2} is a hydrogen atom, a fluorine atom, or a fluorinated alkyl group), or a combination thereof.

As the component generated from the thermal acid generator [A1], sulfonic acids are preferable, fluorinated alkylsulfonic acids having 1 to 10 carbon atoms and sulfonic acids having an alicyclic structure are more preferable, perfluoroalkylsulfonic acids and 10-camphorsulfonic acid are still more preferable, and trifluoromethanesulfonic acid, nonafluorobutanesulfonic acid, and 10-camphorsulfonic acid are particularly preferable.

Examples of the thermal acid generator [A1] include onium salt compounds such as iodonium salt compounds, organic sulfonic acid alkyl esters, 2,4,4,6-tetrabromocyclohexadienone, benzoin tosylate, and 2-nitrobenzyl tosylate.

Examples of the iodonium salt compound include salt compounds of anions such as trifluoromethanesulfonate, nonafluoro-n-butanesulfonate, 10-camphorsulfonate, pyrenesulfonate, n-dodecylbenzenesulfonate, and naphthalenesulfonate and iodonium cations such as diphenyliodonium and bis(4-t-butylphenyl)iodonium.

As the thermal acid generator [A1], onium salt compounds are preferable, iodonium salt compounds are more preferable, and bis(4-t-butylphenyl)iodonium trifluoromethanesulfonate, bis(4-t-butylphenyl)iodonium nonafluoro-n-butanesulfonate, and bis(4-t-butylphenyl)iodonium 10-camphorsulfonate are still more preferable.

When the composition for forming a resist underlayer film includes the thermal acid generator [A1], the lower limit of the content of the thermal acid generator [A1] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 12% by mass, and particularly preferably 10% by mass.

### [Thermally Acid-Generating Polymer [A2]]

The thermally acid-generating polymer [A2] is an organic polymer that generates a component having an acid group (a) by the action of heat. The component generated from the thermally acid-generating polymer [A2] may be either a low molecular weight compound having an acid group (a) or an organic polymer having an acid group (a), but an organic polymer having an acid group (a) is preferable.

The lower limit of the Mw of the thermally acid-generating polymer [A2] is preferably 1,600, more preferably 2,000, and still more preferably 2,500. The upper limit of the Mw is preferably 50,000, more preferably 30,000, and still more preferably 15,000.

Examples of the thermally acid-generating polymer [A2] include a polymer having a structural unit in which one or a plurality of thermal acid generators [A1] are incorporated, and a structural unit having an alkoxysulfonyl group is preferable. Examples of the alkoxysulfonyl group include an alkoxysulfonyl group having 1 to 20 carbon atoms, and an ethoxysulfonyl group is preferable. As the structural unit containing an alkoxysulfonyl group, a styrene-based structural unit containing an aromatic ring substituted with an alkoxysulfonyl group is preferable, and a structural unit represented by the formula given below is more preferable. The thermally acid-generating polymer [A2] may have a structural unit other than the structural unit in which the thermal acid generator [A1] is incorporated.

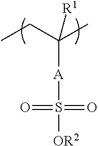

In the above formula, R¹ is a hydrogen atom, a fluorine atom, a methyl group, or a trifluoromethyl group. A is a single bond, an alkylene group having 1 to 10 carbon atoms, a cycloalkylene group having 4 to 20 carbon atoms, an arylene group having 6 to 20 carbon atoms, or a divalent hydrocarbon group composed of a combination thereof. R² is an alkyl group having 1 to 20 carbon atoms.

The lower limit of the content of the structural unit in which the thermal acid generator [A1] is incorporated in all the structural units constituting the thermally acid-generating polymer [A2] is preferably 1 mol %, and more preferably 5 mol %. The upper limit of the content of the structural unit is preferably 80 mol %, and more preferably 60 mol %.

The thermally acid-generating polymer [A2] may have a structural unit other than the structural unit in which the thermal acid generator [A1] is incorporated. The structural unit is not particularly limited, and examples thereof include the same structural units as those constituting each resin in the organic polymer [D1] described later.

The lower limit of the content of other structural unit described above in all the structural units constituting the thermally acid-generating polymer [A2] is preferably 5 mol %, and more preferably 10 mol %. The upper limit of the content of the structural unit is preferably 80 mol %, and more preferably 50 mol %.

When the composition for forming a resist underlayer film includes the thermally acid-generating polymer [A2], the lower limit of the content of the thermally acid-generating polymer [A2] in the components other than the solvent in the composition for forming a resist underlayer film is preferably 80% by mass, more preferably 90% by mass, and still more preferably 95% by mass. The upper limit of the content may be 100% by mass.

### (Photo-Acid Generator [A3])

The photo-acid generator [A3] is a component that generates an acid by the action of radiation. The photo-acid generator [A3] may be used singly or two or more kinds thereof may be used in combination.

As the acid generated from the photo-acid generator [A3], sulfonic acids are preferable, fluorinated alkylsulfonic acids having 1 to 10 carbon atoms and sulfonic acids having an alicyclic structure are more preferable, perfluoroalkylsulfonic acids and 10-camphorsulfonic acid are still more preferable, and trifluoromethanesulfonic acid, nonafluorobutanesulfonic acid and 10-camphorsulfonic acid are particularly preferable.

Examples of the photo-acid generator [A3] include an onium salt compound, an N-sulfonyloxyimide compound, a halogen-containing compound, and a diazoketone compound.

Examples of the onium salt compound include a sulfonium salt, a tetrahydrothiophenium salt, an iodonium salt, a phosphonium salt, a diazonium salt, and a pyridinium salt.

Examples of the anion of the onium salt compound include an anion represented by the following formula.

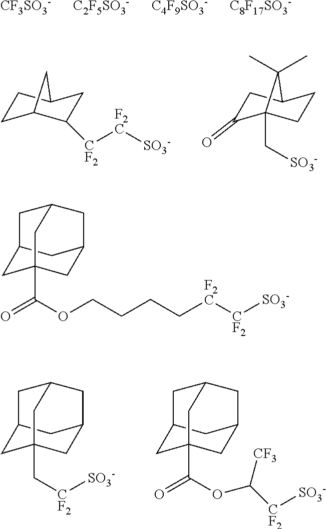

Examples of the cation of the onium salt compound include a cation represented by the following formula.

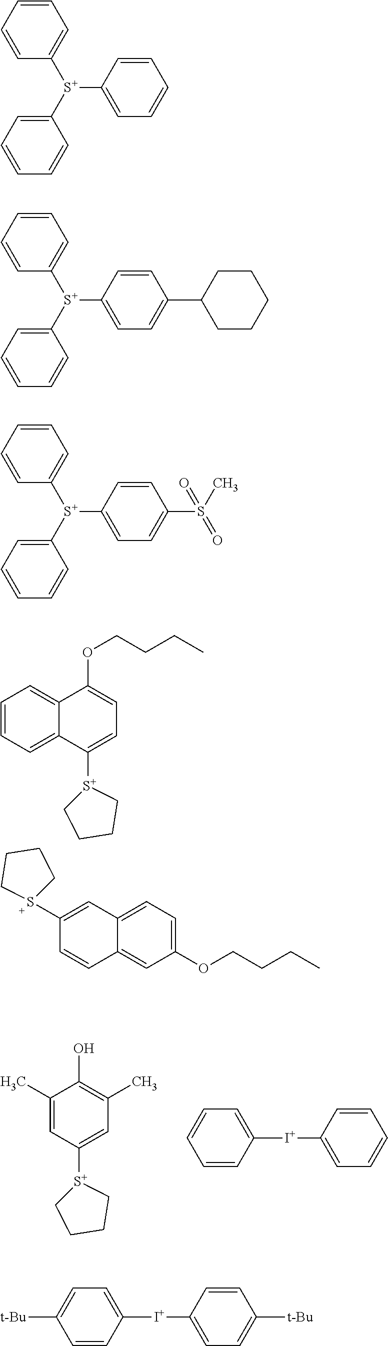

As the onium salt compound, a compound obtained by appropriately combining the anion described above and the cation described above can be used, for example.

Examples of the N-sulfonyloxyimide compound include a compound represented by the following formula.

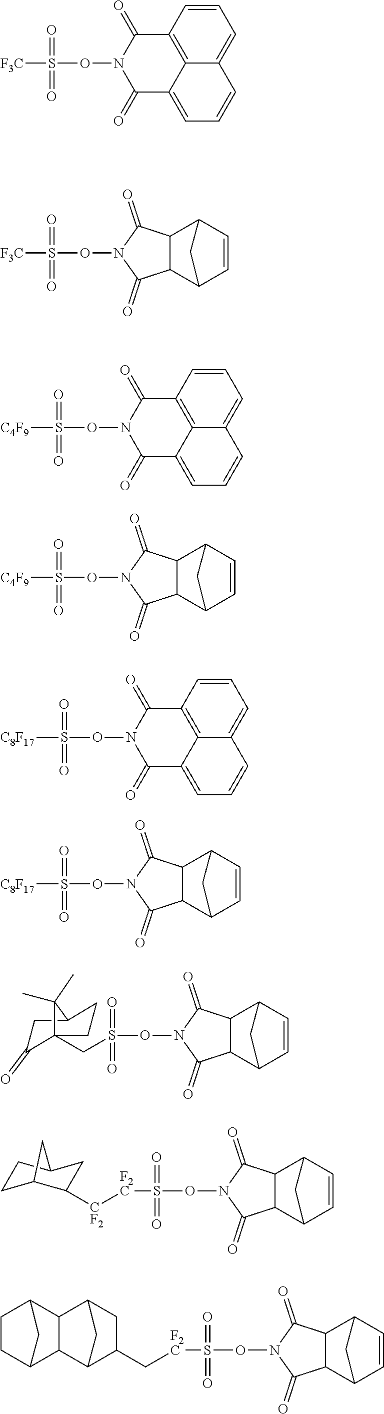

As the photo-acid generator [A3], onium salt compounds are preferable, sulfonium salts are more preferable, and triphenylsulfonium trifluoromethanesulfonate, triphenylsulfonium nonafluorobutanesulfonate, and triphenylsulfonium camphorsulfonate are still more preferable.

When the composition for forming a resist underlayer film includes the photo-acid generator [A3], the lower limit of the content of the photo-acid generator [A3] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 12% by mass, and particularly preferably 10% by mass.

### (Acid Group-Containing Component [B])

The acid group-containing component [B] is a component having an acid group (a) other than the acid generating component [A]. The acid group-containing component [B] may be either a low molecular weight compound (hereinafter, also referred to as an acid group-containing compound [B1]) or an organic polymer (hereinafter, also referred to as an acid group-containing polymer [B2]). The acid group-containing component [B] may be used singly or two or more kinds thereof may be used in combination.

### [Acid Group-Containing Compound [B1]]

The acid group-containing compound [B1] is a low molecular weight compound having an acid group (a). Specific examples of the acid group-containing compound [B1] include the same components as the component having an acid group (a) generated from the thermal acid generator [A1] described above.

When the composition for forming a resist underlayer film includes the acid group-containing compound [B1], the lower limit of the content of the acid group-containing compound [B1] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 10% by mass, and particularly preferably 8% by mass.

### [Acid Group-Containing Polymer [B2]]

The acid group-containing polymer [B2] is an organic polymer having an acid group (a). Examples of the acid group-containing polymer [B2] include an ion exchange resin having a structural unit containing an acid group (a).

The lower limit of the Mw of the acid group-containing polymer [B2] is preferably 1,600, more preferably 2,000, and still more preferably 2,500. On the other hand, the upper limit of the Mw is preferably 50,000, more preferably 30,000, and still more preferably 15,000.

Examples of the ion exchange resin include a polymer obtained by introducing an acid group (a) into an organic polymer such as a styrene-based polymer, a (meth)acrylic polymer, a polyester-based polymer, cellulose or polytetrafluoroethylene. More specific examples thereof include a polymer obtained by sulfonating a novolac resin, a polymer obtained by sulfonating a resol resin, a polymer obtained by sulfonating a styrene polymer crosslinked with divinylbenzene, and a polymer obtained by carboxylating a (meth)acrylic polymer crosslinked with divinylbenzene. Examples of the novolac resin and the resol resin to be sulfonated in the ion exchange resin include the same resins as the novolac resin and the resol resin in the organic polymer [D1] described later.

The structural unit containing an acid group (a) is preferably one obtained by introducing a sulfo group into the structural unit of the novolac resin. Examples of such a structural unit include a structural unit represented by the following formula.

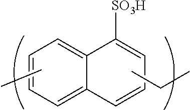

The lower limit of the content of the structural unit containing an acid group (a) in all the structural units constituting the acid group-containing polymer [B2] is preferably 5 mol %, and more preferably 10 mol %. On the other hand, the upper limit of the content of the structural unit is preferably 80 mol %, and more preferably 50 mol %.

The lower limit of the content of the structural unit containing no acid group (a) in all the structural units constituting the acid group-containing polymer [B2] is preferably 5 mol %, and more preferably 10 mol %. On the other hand, the upper limit of the content of the structural unit is preferably 80 mol %, and more preferably 50 mol %.

When the composition for forming a resist underlayer film includes the acid group-containing polymer [B2], the lower limit of the content of the acid group-containing polymer [B2] in the components other than the solvent in the composition for forming a resist underlayer film is preferably 80% by mass, more preferably 90% by mass, and still more preferably 95% by mass. The upper limit of the content may be 100% by mass.

### (Photo-Base Generator [C1])

The photo-base generator [C1] is a component that generates a base by the action of radiation. Examples of the base generated from the photo-base generator [C] include amines such as primary amines, secondary amines, and tertiary amines. The photo-base generator [C1] may be used singly or two or more kinds thereof may be used in combination.

Examples of the photo-base generator [C1] include transition metal complexes of cobalt or the like, orthonitrobenzylcarbamates, α,α-dimethyl-3,5-dimethoxybenzylcarbamates, acyloxyiminos, and acetophenone-based compounds.

Examples of the transition metal complexes of cobalt include the compounds described in paragraph [0198] of JP-A-2017-009673.

Examples of the orthonitrobenzylcarbamates include [[(2-nitrobenzyl)oxy]carbonyl]methylamine, [[(2-nitrobenzyl)oxy]carbonyl]propylamine, [[(2-nitrobenzyl)oxy]carbonyl]hexylamine, [[[(2-nitrobenzyl)oxy]carbonyl]cyclohexylamine, [[(2-nitrobenzyl)oxy]carbonyl]aniline, [[[(2-nitrobenzyl)oxy]carbonyl]piperidine, bis[[(2-nitrobenzyl)oxy]carbonyl]hexamethylenediamine, bis[[(2-nitrobenzyl)oxy]carbonyl]phenylenediamine, bis[[(2-nitrobenzyl)oxy]carbonyl]toluenediamine, bis[[(2-nitrobenzyl)oxy]carbonyl]diaminodiphenylmethane, bis[[(2-nitrobenzyl)oxy]carbonyl]piperazine, [[(2,6-dinitrobenzyl)oxy]carbonyl]methylamine, [[(2,6-dinitrobenzyl)oxy]carbonyl]propylamine, [[(2,6-dinitrobenzyl)oxy]carbonyl]hexylamine, [[(2,6-dinitrobenzyl)oxy]carbonyl]cyclohexylamine, [[[(2,6-dinitrobenzyl)oxy]carbonyl]aniline, [[(2,6-dinitrobenzyl)oxy]carbonyl]piperidine, bis[[(2,6-dinitrobenzyl)oxy]carbonyl]hexamethylenediamine, bis[[(2,6-dinitrobenzyl)oxy]carbonyl]phenylenediamine, bis[[(2,6-dinitrobenzyl)oxy]carbonyl]toluenediamine, bis[[(2,6-dinitrobenzyl)oxy]carbonyl]diaminodiphenylmethane, and bis[[[(2,6-dinitrobenzyl)oxy]carbonyl]piperazine.

Examples of the α,α-dimethyl-3,5-dimethoxybenzylcarbamates include [[(α,α-dimethyl-3,5-dimethoxybenzyl)oxy]carbonyl]methylamine, [[(α,α-dimethyl-3,5-dimethoxybenzyl)oxy]carbonyl]propylamine, [[(α,α-dimethyl-3,5-dimethoxybenzyl)oxy]carbonyl]hexylamine, [[(α,α-dimethyl-3,5-dimethoxybenzyl)oxy]carbonyl]cyclohexylamine, [[(α,α-dimethyl-3,5-dimethoxybenzyl)oxy]carbonyl]aniline, [[(α,α-dimethyl-3,5-dimethoxybenzyl)oxy]carbonyl]piperidine, bis[[(α,α-dimethyl-3,5-dimethoxybenzyl)oxy]carbonyl]hexamethylenediamine, bis[[(α,α-dimethyl-3,5-dimethoxybenzyl)oxy]carbonyl]phenylenediamine, bis[[(α,α-dimethyl-3,5-dimethoxybenzyl)oxy]carbonyl]toluenediamine, bis[[(α,α-dimethyl-3,5-dimethoxybenzyl)oxy]carbonyl]diaminodiphenylmethane, and bis[[(α,α-dimethyl-3,5-dimethoxybenzyl)oxy]carbonyl]piperazine.

Examples of the acyloxyiminos include propionylacetophenone oxime, propionylbenzophenone oxime, propionylacetone oxime, butyrylacetophenone oxime, butyrylbenzophenone oxime, butyrylacetone oxime, adipoylacetophenone oxime, adipoylbenzophenone oxime, adipoylacetone oxime, acryloylacetophenone oxime, acryloylbenzophenone oxime, and acryloylacetone oxime.

Examples of the acetophenone-based compounds include acetophenone-based compounds having an α-aminoketone structure such as 2-benzyl-2-dimethylamino-1-(4-morpholinophenyl)butan-1-one, 2-dimethylamino-2-(4-methylbenzyl)-1-(4-morpholin-4-yl-phenyl)-butan-1-one, and 2-methyl-1-[4-(methylthio)phenyl]-2-morpholinopropan-1-one.

Examples of the photo-base generator [C1] include, besides the examples of the compound recited above, 2-nitrobenzylcyclohexylcarbamate, O-carbamoylhydroxyamide, and O-carbamoylhydroxyamide.

As the photo-base generator [C1], acetophenone-based compounds and 2-nitrobenzylcyclohexylcarbamate are preferable, acetophenone-based compounds having an α-aminoketone structure and 2-nitrobenzylcyclohexylcarbamate are more preferable, and 2-methyl-1-[4-(methylthio)phenyl]-2-morpholinopropan-1-one and 2-benzyl-2-dimethylamino-1-(4-morpholinophenyl)butan-1-one are still more preferable.

### (Base-Containing Component [C2])

Examples of the base-containing component [C2] include onium salt compounds that are not decomposed by the action of heat such as sulfonium salt compounds, and amines.

Examples of the sulfonium salt compounds include a compound represented by the following formula.

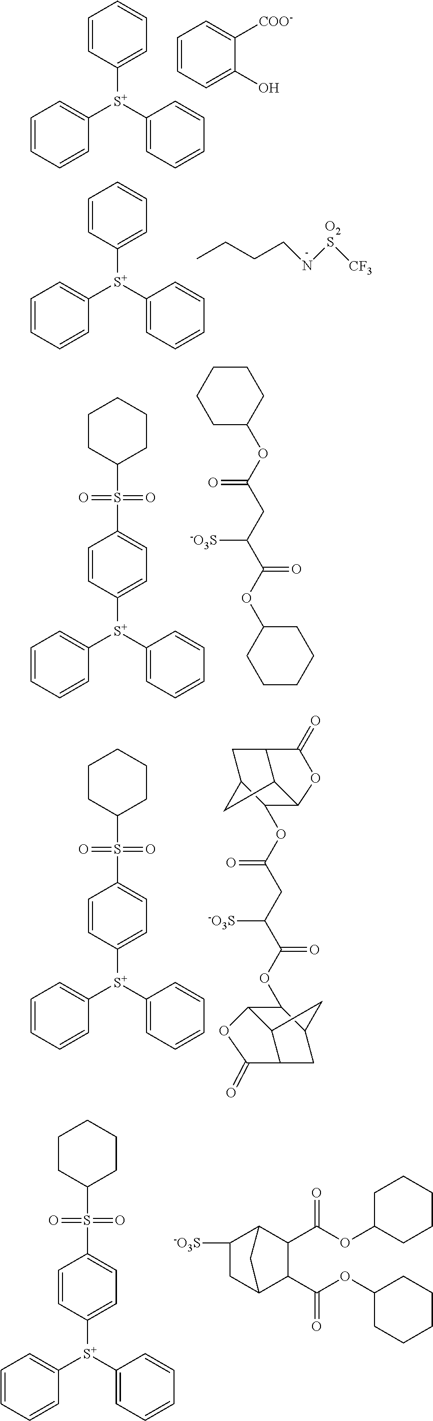

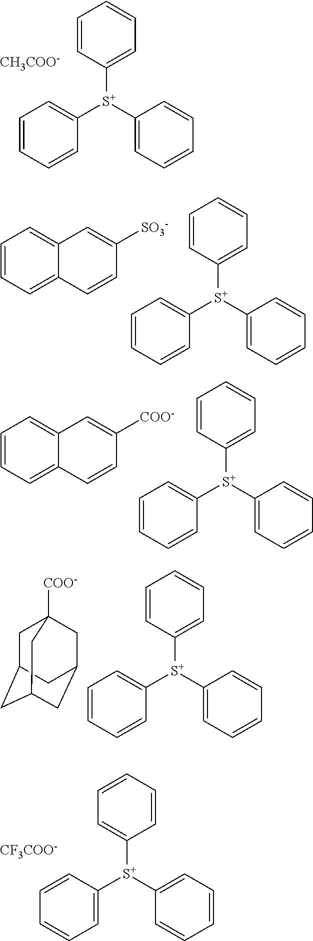

Examples of the amines include aliphatic amines, aromatic amines, heterocyclic amines, quaternary ammonium hydroxides, and quaternary ammonium salts of carboxylic acids.

Examples of the aliphatic amines include aliphatic amines such as trimethylamine, diethylamine, triethylamine, di-n-propylamine, tri-n-propylamine, di-n-pentylamine, tri-n-pentylamine, diethanolamine, triethanolamine, dicyclohexylamine, and dicyclohexylmethylamine.

Examples of the aromatic amines include aniline, benzylamine, N,N-dimethylaniline, and diphenylamine.

Examples of the heterocyclic amines include pyridine, 2-methylpyridine, 4-methylpyridine, 2-ethylpyridine, 4-ethylpyridine, 2-phenylpyridine, 4-phenylpyridine, N-methyl-4-phenylpyridine, 4-dimethylaminopyridine, imidazole, benzimidazole, 4-methylimidazole, 2-phenylbenzimidazole, 2,4,5-triphenylimidazole, nicotine, nicotinic acid, nicotinic acid amide, quinoline, 8-oxyquinoline, pyrazine, pyrazole, pyridazine, purine, pyrrolidine, piperidine, piperazine, morpholine, 4-methylmorpholine, 1,5-diazabicyclo[4,3,0]-5-nonene, and 1,8-diazabicyclo[5,3,0]-7-undecene.

Examples of the quaternary ammonium hydroxides include tetramethylammonium hydroxide, tetraethylammonium hydroxide, tetra-n-butylammonium hydroxide, and tetra-n-hexylammonium hydroxide.

Examples of the quaternary ammonium salts of carboxylic acids include tetramethylammonium acetate, tetramethylammonium benzoate, tetra-n-butylammonium acetate, and tetra-n-butylammonium benzoate.

When the composition for forming a resist underlayer film includes the photo-base generator [C1] or the base-containing component [C2], the lower limit of the content of the photo-base generator [C1] or the base-containing component [C2] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 10% by mass, and particularly preferably 8% by mass.

The composition for forming a resist underlayer film may further include an organic polymer other than the acid group-containing component [B] (hereinafter, also referred to as “organic polymer [D1]”), an inorganic polymer [D2], an aromatic ring-containing compound [D3], an additive [D4], and so on.

### (Organic Polymer [D1])

As the organic polymer [D1], for example, those described in paragraphs [0040] to [0116] of JP-A-2016-206676 and the like can be used, but from the viewpoint of further improving the etching resistance of the underlayer film, a novolac resin, a resol resin, an aromatic ring-containing vinyl resin, an acenaphthylene resin, an indene resin, a polyarylene resin, a triazine resin, a calixarene resin, a fullerene resin, and a pyrene resin are preferable, and a novolac resin and an acenaphthylene resin are more preferable.

The lower limit of the Mw of the novolac resin, the resol resin, the aromatic ring-containing vinyl resin, the acenaphthylene resin, the indene resin, the polyarylene resin, the triazine resin, the fullerene resin, or the pyrene resin is preferably 500, more preferably 1,000, and still more preferably 2,000. On the other hand, the upper limit of the Mw is preferably 10,000. The lower limit of the ratio of the Mw to the Mn (Mw/Mn) of these resins is preferably 1.1. The upper limit of the Mw/Mn is preferably 5, more preferably 3, and still more preferably 2. By setting the Mw and the Mw/Mn within the above ranges, the flatness and the surface coatability of the underlayer film can be improved.

The lower limit of the molecular weight of the calixarene resin is preferably 500, more preferably 700, and still more preferably 1,000 from the viewpoint of improving the flatness of the resist underlayer film. The upper limit of the molecular weight is preferably 5,000, more preferably 3,000, and still more preferably 1,500. When the calixarene resin has a molecular weight distribution, the molecular weight of the calixarene resin means a polystyrene-equivalent Mw determined by GPC.

### (Inorganic Polymer [D2])

Examples of the inorganic polymer [D2] include polysiloxane [D2-1], a complex (polynuclear complex) [D2-2] containing a plurality of metal atoms, an oxygen atom bridging between the metal atoms (hereinafter, also referred to as “bridging oxygen atom”), and a multidentate ligand coordinated to the metal atoms, and polycarbosilane [D2-3].

### [Polysiloxane [D2-1]]

Examples of the polysiloxane [D2-1] include those having a structural unit (I) represented by the following formula (I) and/or a structural unit (II) represented by the following formula (II). Each structural unit in the polysiloxane [D2-1] can be used singly or two or more kinds thereof may be used in combination.

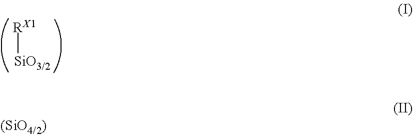

in the formula (I), R^{X1} is a monovalent organic group having 1 to 20 carbon atoms.

Here, the “organic group” refers to a group having at least one carbon atom.

As the monovalent organic group represented by R^{X1}, a monovalent hydrocarbon group, a monovalent fluorinated hydrocarbon group, or a monovalent group (a) having a divalent heteroatom-containing group between two adjacent carbon atoms of a monovalent hydrocarbon group is preferable, a monovalent chain hydrocarbon group, a monovalent aromatic hydrocarbon group, a monovalent fluorinated aromatic hydrocarbon group, or a group containing a heterocyclic ring is more preferable, and an alkyl group, an aryl group, a fluoroaryl group, or a group containing a nitrogen-containing heterocyclic ring is still more preferable. Examples of the nitrogen-containing heterocyclic ring include an azocycloalkane ring and an isocyanuric ring.

Examples of the structural unit (I) include a structural unit represented by the following formula.

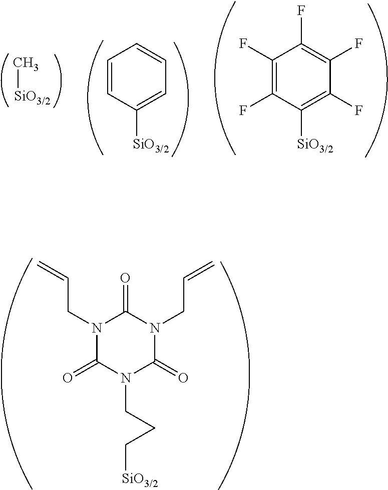

The lower limit of the content of the structural unit (I) in the polysiloxane [D2-1] is preferably 1 mol %, and more preferably 5 mol %. On the other hand, the upper limit of the content of the structural unit (I) is preferably 60 mol %, and more preferably 40 mol %.

The lower limit of the content of the structural unit (II) in the polysiloxane [D2-1] is preferably 40 mol %, and more preferably 60 mol %. The upper limit of the content of the structural unit (II) is preferably 99 mol %, and more preferably 95 mol %.

The lower limit of the Mw of the polysiloxane [D2-1] is preferably 500, more preferably 800, and still more preferably 1,200. The upper limit of the Mw is preferably 100,000, more preferably 30,000, still more preferably 10,000, and particularly preferably 5,000.

### [Complex [D2-2]]

As the metal atom in the complex [D2-2], titanium, tantalum, zirconium, and tungsten (hereinafter, these are also referred to as “specific metal atoms”) are preferable, and titanium and zirconium are more preferable. These metal atoms may be used singly or two or more kinds thereof may be used in combination.

The complex [D2-2] may be a stable polynuclear complex by containing a bridging oxygen atom. A plurality of bridging oxygen atoms may be bonded to one metal atom, but for some metal atoms, only one bridging oxygen atom may be bonded to one metal atom. Preferably, the complex [D2-2] mainly contains a structure in which two bridging oxygen atoms are bonded to one metal atom. Here, “mainly contain” means that among all the metal atoms constituting the complex [D2-2], metal atoms in a proportion of 50 mol % or more, preferably 70 mol % or more, more preferably 90 mol % or more, and particularly preferably 95 mol % or more are each bonded to two bridging oxygen atoms.

The complex [D2-2] may have another bridging ligand such as a peroxide ligand (—O—O—) in addition to the bridging oxygen atom.

The multidentate ligand in the complex [D2-2] improves the solubility of the complex [D2-2], thereby improving the removability of the underlayer film. As the multidentate ligand, a hydroxyacid ester, a β-diketone, a β-ketoester, a malonic diester in which a carbon atom at the α-position is optionally substituted (hereinafter, also referred to as a “malonic diester”), a hydrocarbon having a n bond, or a ligand derived from such a compound is preferable. Each of these compounds usually forms a multidentate ligand as an anion formed by obtaining one electron, forms a multidentate ligand as an anion resulting from elimination of a proton, or forms a multidentate ligand as it is.

The lower limit of the molar ratio of the multidentate ligand to the metal atom (multidentate ligand/metal atom) in the complex [D2-2] is preferably 1, more preferably 1.5, and still more preferably 1.8. The upper limit of the ratio is preferably 3, more preferably 2.5, and still more preferably 2.2.

The complex [D2-2] may contain another ligand besides the above-described bridging ligand and multidentate ligand.

### [Polycarbosilane [D2-3]]

The polycarbosilane [D2-3] is a polymer having a Si—C bond in the main chain.

The polycarbosilane [D2-3] has, for example, a first structural unit represented by the following formula (1) (hereinafter, also referred to as “structural unit (i)”). The polycarbosilane [D2-3] may have a second structural unit represented by the formula (2) (hereinafter, also referred to as “structural unit (ii)”) and a third structural unit represented by the formula (3) (hereinafter, also referred to as “structural unit (iii)”), both described later. The polycarbosilane [D2-3] may be used singly or two or more kinds thereof may be used in combination.

### (Structural Unit (i))

The structural unit (i) is represented by the following formula (1),

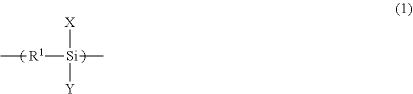

In the formula (1), R¹ is a substituted or unsubstituted divalent hydrocarbon group having 1 to 20 carbon atoms. X and Y each independently represent a hydrogen atom, a hydroxy group, a halogen atom, or a monovalent organic group having 1 to 20 carbon atoms.

Examples of R¹ in the formula (1) include a substituted or unsubstituted divalent chain hydrocarbon group having 1 to 20 carbon atoms, a substituted or unsubstituted divalent alicyclic hydrocarbon group having 3 to 20 carbon atoms, and a substituted or unsubstituted divalent aromatic hydrocarbon group having 6 to 20 carbon atoms. In the present description, the chain hydrocarbon group includes both a linear hydrocarbon group and a branched hydrocarbon group.

Examples of the unsubstituted divalent chain hydrocarbon group having 1 to 20 carbon atoms include chain saturated hydrocarbon groups such as a methanediyl group and an ethanediyl group, and chain unsaturated hydrocarbon groups such as an ethenediyl group and a propenediyl group.

Examples of the unsubstituted divalent alicyclic hydrocarbon group having 3 to 20 carbon atoms include monoalicyclic saturated hydrocarbon groups such as a cyclobutanediyl group, monoalicyclic unsaturated hydrocarbon groups such as a cyclobutenediyl group, polyalicyclic saturated hydrocarbon groups such as a bicyclo[2.2.1]heptanediyl group, and polyalicyclic unsaturated hydrocarbon groups such as a bicyclo[2.2.1]heptenediyl group.

Examples of the unsubstituted divalent aromatic hydrocarbon group having 6 to 20 carbon atoms include a phenylene group, a biphenylene group, a phenylene ethylene group, and a naphthylene group.

Examples of the substituent in the substituted divalent hydrocarbon group having 1 to 20 carbon atoms represented by R¹ include a halogen atom, a hydroxy group, a cyano group, a nitro group, an alkoxy group, an acyl group, and an acyloxy group.

As R¹, an unsubstituted chain saturated hydrocarbon group is preferable, and a methanediyl group or an ethanediyl group is more preferable.

Examples of the monovalent organic group having 1 to 20 carbon atoms represented by X or Y in the above formula (1) include a monovalent hydrocarbon group having 1 to 20 carbon atoms, a monovalent group having a divalent heteroatom-containing group between two adjacent carbon atoms of the aforementioned hydrocarbon group, and a monovalent group resulting from replacement of some or all hydrogen atoms of the aforementioned hydrocarbon group or the aforementioned group containing a divalent heteroatom-containing group by a monovalent heteroatom-containing group.

Examples of the monovalent hydrocarbon group having 1 to 20 carbon atoms include a monovalent chain hydrocarbon group having 1 to 20 carbon atoms, a monovalent alicyclic hydrocarbon group having 3 to 20 carbon atoms, and a monovalent aromatic hydrocarbon group having 6 to 20 carbon atoms.

Examples of the monovalent chain hydrocarbon group having 1 to 20 carbon atoms include alkyl groups such as a methyl group and an ethyl group, alkenyl groups such as an ethenyl group, and alkynyl groups such as an ethynyl group.

Examples of the monovalent alicyclic hydrocarbon group having 3 to 20 carbon atoms include monovalent monoalicyclic saturated hydrocarbon groups such as a cyclopentyl group and a cyclohexyl group, monovalent monoalicyclic unsaturated hydrocarbon groups such as a cyclopentenyl group and a cyclohexenyl group, monovalent polyalicyclic saturated hydrocarbon groups such as a norbornyl group, and an adamantyl group, and monovalent polyalicyclic unsaturated hydrocarbon groups such as a norbornenyl group and a tricyclodecenyl group.

Examples of the monovalent aromatic hydrocarbon group having 6 to 20 carbon atoms include aryl groups such as a phenyl group, a tolyl group, a xylyl group, a naphthyl group, a methylnaphthyl group, and an anthryl group, and aralkyl groups such as a benzyl group, a phenethyl group, a naphthylmethyl group, and an anthrylmethyl group.

Examples of heteroatoms that constitute divalent or monovalent heteroatom-containing groups include an oxygen atom, a nitrogen atom, a sulfur atom, a phosphorus atom, a silicon atom, and halogen atoms. Examples of the halogen atoms include a fluorine atom, a chlorine atom, a bromine atom, and an iodine atom.

Examples of the divalent heteroatom-containing group include —O—, —CO—, —S—, —CS—, —NR′—, and groups in which two or more of the foregoing are combined. R′ is a hydrogen atom or a monovalent hydrocarbon group.

Examples of the monovalent heteroatom-containing group include halogen atoms such as a fluorine atom, a chlorine atom, a bromine atom, and an iodine atom; a hydroxyl group, a carboxyl group, a cyano group, an amino group, and a sulfanyl group.

As the monovalent organic group having 1 to 20 carbon atoms represented by X or Y, a monovalent hydrocarbon group is preferable, a monovalent chain hydrocarbon group or a monovalent aromatic hydrocarbon group is more preferable, and an alkyl group or an aryl group is still more preferable.

The number of the carbon atoms of the monovalent organic group represented by X or Y is preferably 1 to 10, and more preferably 1 to 6.

Examples of the halogen atom represented by X or Y include a fluorine atom, a chlorine atom, a bromine atom, and an iodine atom. As the halogen atom, a chlorine atom or a bromine atom is preferable.

When the polycarbosilane [D2-3] has a structural unit (I), the lower limit of the content of the structural unit (I) relative to all the structural units constituting the polycarbosilane [D2-3] is preferably 5 mol %, more preferably 30 mol %, still more preferably 60 mol %, and particularly preferably 80 mol %. The upper limit of the content of the structural unit (I) may be 100 mol %. The content (mol %) of each structural unit of the polycarbosilane [D2-3] is usually equivalent to the molar ratio of the monomer to give each structural unit used for the synthesis of the polycarbosilane [D2-3].

(Structural Unit (ii))

The structural unit (ii) is an arbitrary structural unit which the polycarbosilane [D2-3] may have, and is represented by the following formula (2).

(SiO_{4/2}) (2)

When the polycarbosilane [D2-3] has a structural unit (ii), the lower limit of the content of the structural unit (ii) relative to all the structural units constituting the polycarbosilane [D2-3] is preferably 0.1 mol %, more preferably 1 mol %, and still more preferably 5 mol %. The upper limit of the content of the structural unit (ii) is preferably 50 mol %, more preferably 40 mol %, still more preferably 30 mol %, and particularly preferably 20 mol %.

(Structural Unit (iii))

The structural unit (iii) is an arbitrary structural unit which the polycarbosilane [D2-3] may have, and is represented by the following formula (3).

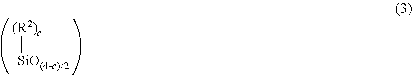

In the formula (3), R² is a substituted or unsubstituted monovalent hydrocarbon group having 1 to 20 carbon atoms. c is 1 or 2. When c is 2, two R²'s are the same or different from each other.

As c, 1 is preferable.

Examples of R² include the same groups as the monovalent hydrocarbon groups having 1 to 20 carbon atoms recited as examples for X and Y of the above formula (1). Examples of the substituent of the monovalent hydrocarbon group having 1 to 20 carbon atoms include the same groups as the monovalent heteroatom-containing groups recited as examples for X and Y of the above formula (1).

As R², a substituted or unsubstituted monovalent chain hydrocarbon group or a substituted or unsubstituted monovalent aromatic hydrocarbon group is preferable, an alkyl group or an aryl group is more preferable, and a methyl group or a phenyl group is still more preferable.

When the polycarbosilane [D2-3] has a structural unit (iii), the lower limit of the content of the structural unit (iii) relative to all the structural units constituting the polycarbosilane [D2-3] is preferably 0.1 mol %, more preferably 1 mol %, and still more preferably 5 mol %. The upper limit of the content of the structural unit (iii) is preferably 50 mol %, more preferably 40 mol %, still more preferably 30 mol %, and particularly preferably 20 mol %.

### (Aromatic Ring-Containing Compound [D3])

The aromatic ring-containing compound [D3] is a compound having an aromatic ring and having a molecular weight of 600 or more and 3,000 or less (excluding the organic polymer [D1] and the inorganic polymer [D2]). When the aromatic ring-containing compound [D3] has a molecular weight distribution, the molecular weight of the aromatic ring-containing compound [D3] is, for example, a polystyrene-equivalent weight-average molecular weight (Mw) measured by GPC. When the composition for forming a resist underlayer film includes the aromatic ring-containing compound [D3], the heat resistance and etching resistance of the underlayer film can be improved as in the case of containing the organic polymer [D1] having an aromatic ring. Specific examples of the aromatic ring-containing compound [D3] include the compounds described in paragraphs [0117] to [0179] of JP-A-2016-206676.

### (Additive [D4])

Examples of the additive [D4] include a crosslinking agent [D4-1], a crosslinking accelerator [D4-2], and a surfactant. The composition for forming a resist underlayer film preferably further includes the crosslinking agent [D4-1] and/or the crosslinking accelerator [D4-2].

### [Crosslinking Agent [D4-1]]

The crosslinking agent [D4-1] is a component that forms a crosslinking bond between organic polymers [D1] or the like by the action of heat or the like. When the composition for forming a resist underlayer film includes the crosslinking agent [D4-1], the hardness of the underlayer film can be improved.

Examples of the crosslinking agent [D4-1] include a compound having an alkoxyalkylated amino group and a hydroxymethyl group-substituted phenol compound.

Examples of the hydroxymethyl group-substituted phenol compound include 2-hydroxymethyl-4,6-dimethylphenol, 1,3,5-trihydroxymethylbenzene, 3,5-dihydroxymethyl-4-methoxytoluene[2,6-bis(hydroxymethyl)-p-cresol], 4,4′-(1-(4-(1-(4-hydroxy-3,5-bis(methoxymethyl)phenyl)-1-methylethyl)phenyl)ethylidene)bis (2,6-bis(methoxymethyl)phenol), and 5,5′-(1-methylethylidene)bis(2-hydroxy-1,3-benzenedimethanol).

Examples of the compound having an alkoxyalkylated amino group include a compound derived from a nitrogen-containing compound having a plurality of active methylol groups in one molecule, such as (poly)methylol melamine, (poly)methylol glycoluril, (poly)methylol benzoguanamine, and (poly)methylol urea, by replacing at least some of the hydrogen atoms of the hydroxy group in a methylol group with an alkyl group such as a methyl group or a butyl group. The compound having an alkoxyalkylated amino group may be a mixture obtained by mixing a plurality of substituted compounds, or may contain an oligomer component formed by partial self-condensation.

As the crosslinking agent [D4-1], besides the above-described compounds, for example, a polyfunctional (meth)acrylate compound, an epoxy compound, a hydroxymethyl group-substituted phenol compound, and an alkoxyalkyl group-containing phenol compound can also be used. Specific examples of these compounds include the compounds described in paragraphs [0203] to [0207] of JP-A-2016-206676.

As the crosslinking agent [D4-1], a hydroxymethyl group-substituted phenol compound and a compound having an alkoxyalkylated amino group are preferable, and 5,5′-(1-methylethylidene)bis(2-hydroxy-1,3-benzenedimethanol) and 2,4,6-tris[bis(methoxymethyl)amino]-1,3,5-triazine are more preferable.

When the composition for forming a resist underlayer film includes the crosslinking agent [D4-1], the lower limit of the content of the crosslinking agent [D4-1] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 10% by mass, and particularly preferably 8% by mass.

### [Crosslinking Accelerator [D4-2]]

The crosslinking accelerator [D4-2] accelerates, for example, formation of a crosslinking bond by the crosslinking agent [D4-1], hydrolysis condensation by a hydrolyzable group remaining in the polysiloxane [D2-1], the complex [D2-2], or the like. As the crosslinking accelerator [D4-2], for example, a nitrogen-containing compound having an acid-dissociable group can be used.

Examples of the nitrogen-containing compound having an acid-dissociable group include N-t-butoxycarbonylpiperidine, N-t-butoxycarbonylimidazole, N-t-butoxycarbonylbenzimidazole, N-t-butoxycarbonyl-2-phenylbenzimidazole, N-(t-butoxycarbonyl) di-n-octylamine, N-(t-butoxycarbonyl) diethanolamine, N-(t-butoxycarbonyl) dicyclohexylamine, N-(t-butoxycarbonyl) diphenylamine, N-t-butoxycarbonyl-4-hydroxypiperidine, and N-t-amyloxycarbonyl-4-hydroxypiperidine.

When the composition for forming a resist underlayer film includes the crosslinking accelerator [D4-2], the lower limit of the content of the crosslinking accelerator [D4-2] in the components other than the solvent in the composition for forming a resist underlayer film is preferably 0.1% by mass, more preferably 1% by mass, and still more preferably 2% by mass. The upper limit of the content is preferably 20% by mass, more preferably 15% by mass, still more preferably 10% by mass, and particularly preferably 8% by mass.

The surfactant improves the application surface uniformity of the underlayer film to be formed and inhibits the occurrence of application unevenness. Specific examples of the surfactants that can be used include those described in paragraph [0216] of JP-A-2016-206676.

### (Solvent [E])

Examples of the solvent [E] include a hydrocarbon-based solvent, an ester-based solvent, an alcohol-based solvent, a ketone-based solvent, an ether-based solvent, and a nitrogen-containing solvent. The solvent [E] may be used singly or two or more kinds thereof may be used in combination.

Examples of the hydrocarbon-based solvent include aliphatic hydrocarbon-based solvents such as n-pentane, n-hexane, and cyclohexane, and aromatic hydrocarbon-based solvents such as benzene, toluene, and xylene.

Examples of the ester-based solvent include carbonate-based solvents such as diethyl carbonate, acetic acid monoacetate ester-based solvents such as methyl acetate and ethyl acetate, lactone-based solvents such as γ-butyrolactone, polyhydric alcohol partial ether carboxylate-based solvents such as diethylene glycol monomethyl ether acetate and propylene glycol monomethyl ether acetate, and lactate ester-based solvents such as methyl lactate and ethyl lactate.

Examples of the alcohol-based solvent include monoalcohol-based solvents such as methanol, ethanol, n-propanol, and 4-methyl-2-pentanol, and polyhydric alcohol-based solvents such as ethylene glycol and 1,2-propylene glycol.

Examples of the ketone-based solvent include chain ketone-based solvents such as methyl ethyl ketone and methyl isobutyl ketone, and cyclic ketone-based solvents such as cyclohexanone.

Examples of the ether-based solvent include chain ether-based solvents such as n-butyl ether, polyhydric alcohol ether-based solvents such as cyclic ether-based solvents such as tetrahydrofuran, and polyhydric alcohol partial ether-based solvents such as diethylene glycol monomethyl ether and propylene glycol monomethyl ether.

Examples of the nitrogen-containing solvent include chain nitrogen-containing solvents such as N,N-dimethylacetamide, and cyclic nitrogen-containing solvents such as N-methylpyrrolidone.

As the solvent [E], an alcohol-based solvent, an ether-based solvent, or an ester-based solvent is preferable, a monoalcohol-based solvent, a polyhydric alcohol partial ether-based solvent, or a polyhydric alcohol partial ether carboxylate-based solvent is more preferable, and 4-methyl-2-pentanol, propylene glycol monomethyl ether, or propylene glycol monomethyl ether acetate is still more preferable.

The lower limit of the content ratio of the solvent [E] in the composition for forming a resist underlayer film is preferably 50% by mass, more preferably 60% by mass, and still more preferably 70% by mass. The upper limit of the content is preferably 99.9% by mass, more preferably 99% by mass, and still more preferably 95% by mass.

(Method for Preparing Composition for Forming a Resist Underlayer Film) The composition for forming a resist underlayer film can be prepared by mixing at least one selected from the group consisting of an acid generating component [A], an acid group-containing component [B], a photo-base generator [C1], and a base-containing component [C2], a solvent [E], and optional components as necessary in a prescribed ratio, and then preferably filtering the resulting mixture through a membrane filter having a pore size of 0.5 μm or less or the like.

### EXAMPLES

Hereinbelow, the present invention will specifically be described on the basis of examples, but is not limited to these examples. Methods for measuring various physical property values will be described below.

### [Weight-Average Molecular Weight (Mw)]

The Mw of a polymer was measured by gel permeation chromatography (detector: differential refractometer) with monodisperse polystyrene standards using GPC columns (“G2000HXL”×2 and “G3000HXL”×1) manufactured by Tosoh Corporation under the following analysis conditions: flow rate: 1.0 mL/min; elution solvent: tetrahydrofuran; column temperature: 40° C.

### [Average Thickness of Film]

The average thickness of a film was determined as a value obtained by measuring the film thickness at arbitrary nine points at intervals of 5 cm including the center of the resist underlayer film formed on a 12-inch silicon wafer using a spectroscopic ellipsometer (“M2000D” available from J.A. WOOLLAM Co.) and calculating the average value of the film thicknesses.

### <Preparation of Composition for Forming a Resist Underlayer Film>

The acid generating component [A], the acid group-containing component [B], the photo-base generator [C1], the base-containing component [C2], the organic polymer [D1], the inorganic polymer [D2], the additive [D4], and the solvent [E] that were used for the preparation of the composition for underlayer film formation are given below.

### (Acid Generating Component [A])

Compounds (A-1) to (A-3) as the thermal acid generator [A1] and a thermally acid-generating polymer (A-4) as the thermally acid-generating polymer [A2] are given below.
A-1: Compound represented by the following formula (a-1)
A-2: Compound represented by the following formula (a-2)
A-3: Compound represented by the following formula (a-3)
A-4: Resin represented by the following formula (a-4) (Mw: 3,000)

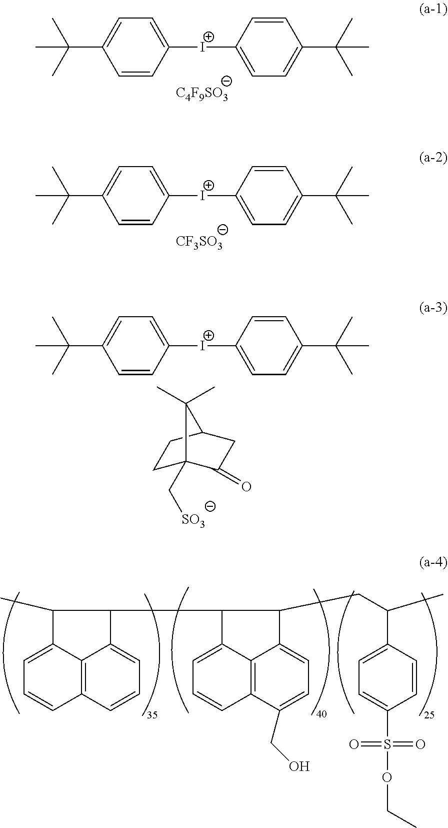

Compounds (A-5) to (A-6) as the photo-acid generator [A3] are given below.
A-5: Compound represented by the following formula (a-5)
A-6: Compound represented by the following formula (a-6)

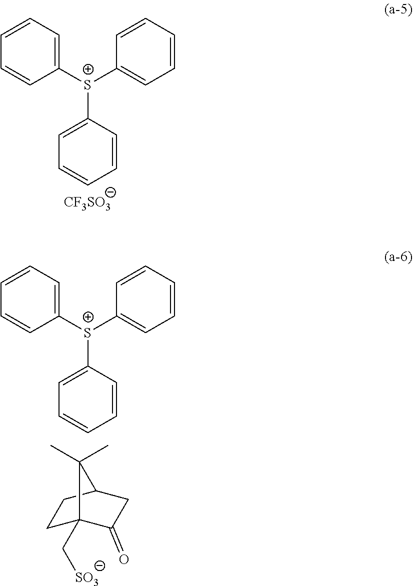

### (Acid Group-Containing Component [B])

The resin (B-1) as the acid group-containing polymer [B2] is given below.
B-1: Acid group-containing polymer represented by the following formula (b-1) (Mw: 3,000)

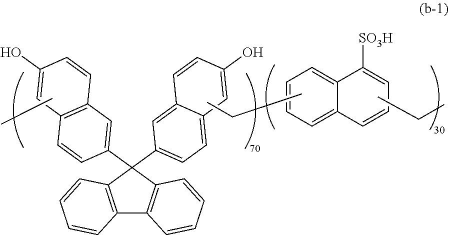

### (Photo-Base Generator [C1])

C1-1: Compound represented by the following formula (c1-1)
C1-2: Compound represented by the following formula (c1-2)
C1-3: Compound represented by the following formula (c1-3)

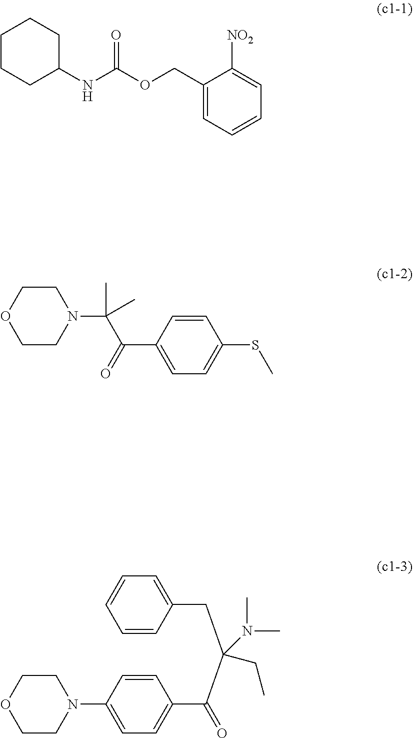 [SMILES: O=C(NC1CCCCC1)OCc1ccccc1[N+](=O)[O-] ; CSc1ccc(C(=O)C(C)(C)N2CCOCC2)cc1 ; CCC(Cc1ccccc1)(C(=O)c1ccc(N2CCOCC2)cc1)N(C)C]

### (Base-Containing Component [C2])

C2-1: Compound represented by the following formula (c2-1)

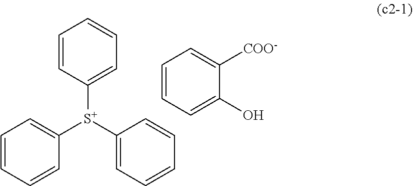

### (Organic Polymer [D1] and Inorganic Polymer [D2])

The organic polymers [D1], (D1-1) to (D1-6), and the inorganic polymers [D2], (D2-1-1) to (D2-1-4) and (D2-2-1) to (D2-2-2), are given below.
D1-1: Organic polymer represented by the following formula (c-1) (Mw: 2,000)
D1-2: Organic polymer represented by the following formula (c-2) (Mw: 1,100)
D1-3: Organic polymer represented by the following formula (c-3) (Mw: 2,000)
D1-4: Organic polymer represented by the following formula (c-4) (Mw: 1,800)
D1-5: Organic polymer represented by the following formula (c-5) (Mw: 2,800)
D1-6: Organic polymer represented by the following formula (c-6) (Mw: 2,000)
D2-1-1: Organic polymer represented by the following formula (c-7) (Mw: 1,500)
D2-1-2: Organic polymer represented by the following formula (c-8) (Mw: 2,000)
D2-1-3: Organic polymer represented by the following formula (c-9) (Mw: 2,000)
D2-1-4: Organic polymer represented by the following formula (c-10) (Mw: 3,000)
D2-2-1: Organic polymer represented by the following formula (c-11) (Mw: 2,500)
D2-2-2: Organic polymer represented by the following formula (c-12) (Mw: 3,000)

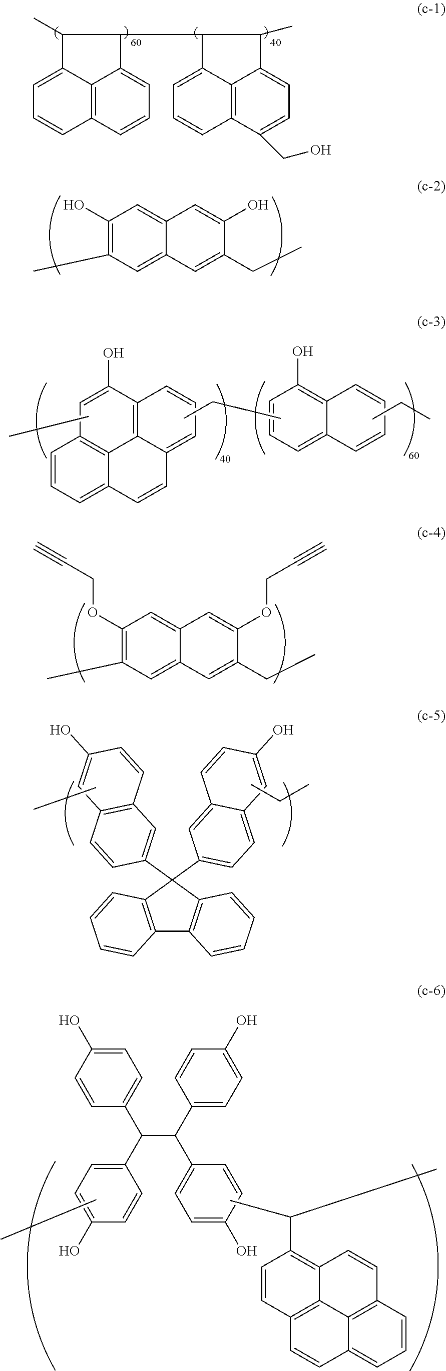

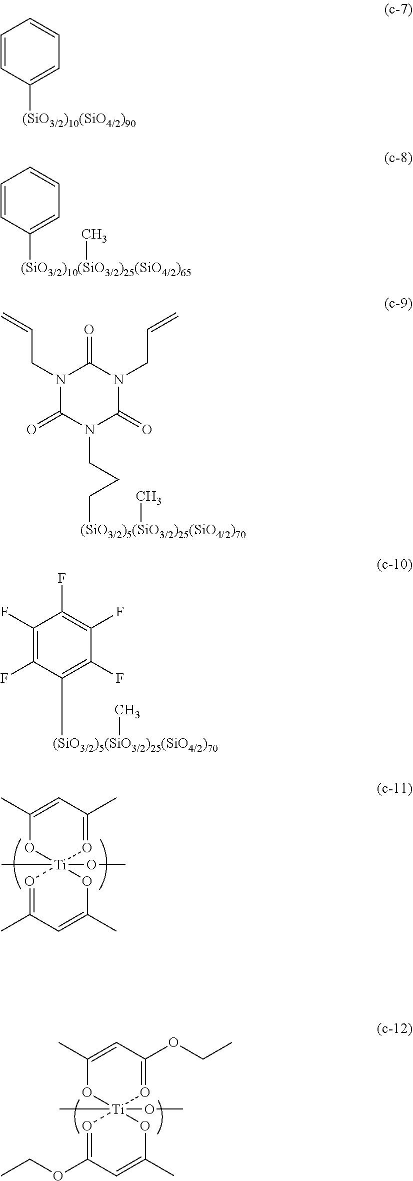

### <Synthesis of Polycarbosilane [D2-3] as Inorganic Polymer [D2]>

The monomers used for the synthesis in this example are given below. In the following Synthesis examples 1 to 10, unless otherwise specified particularly, the term “parts by mass” means a value taken when the total mass of the monomers used or the mass of the solution of polycarbosilane (g) in diisopropyl ether was 100 parts by mass. Herein, mol % means a value taken when the number of moles of all Si in the monomer used is 100 mol %.

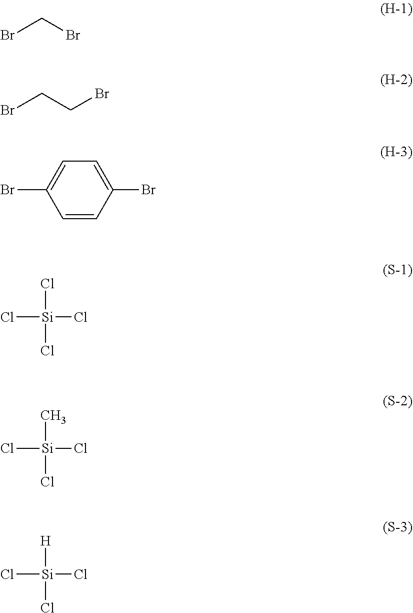

### [Concentration of Polycarbosilane [D2-3] in Solution]

The mass of the residue after calcining 0.5 g of the solution of polycarbosilane [D2-3] at 250° C. for 30 minutes was measured, and the concentration (% by mass) of the polycarbosilane [D2-3] in the solution was calculated by dividing the mass of the residue by the mass of the solution of the polycarbosilane [D2-3].

### (Synthesis of Polycarbosilane (g))

### [Synthesis Example 1] (Synthesis of Polycarbosilane (g-1))

To a reaction vessel purged with nitrogen, magnesium (120 mol %) and tetrahydrofuran (35 parts by mass) were added, and the mixture was stirred at 20° C. Next, the compound represented by the above formula (H-1), the compound represented by the above formula (S-2) and the compound represented by the above formula (S-3) were dissolved in tetrahydrofuran (355 parts by mass) such that the molar ratio was 50/15/35 (mol %) to prepare a monomer solution. The temperature in the reaction vessel was adjusted to 20° C., and the monomer solution was added dropwise thereto over 1 hour with stirring. The completion of the dropwise addition was taken as the start time of a polymerization reaction, and the reaction was performed at 40° C. for 1 hour and then at 60° C. for 3 hours. After the completion of the reaction, tetrahydrofuran (213 parts by mass) was added, and the polymerization solution was ice-cooled to 10° C. or lower. To the polymerization solution cooled was added triethylamine (150 mol %), and then methanol (150 mol %) was added dropwise from a dropping funnel over 10 minutes with stirring. The completion of the dropwise addition was taken as the start time of a reaction, and the reaction was performed at 20° C. for 1 hour. The polymerization solution was poured into diisopropyl ether (700 parts by mass), and a precipitated salt was separated by filtration. Next, tetrahydrofuran, surplus triethylamine, and surplus methanol in the filtrate were removed using an evaporator. The resulting residue was charged into diisopropyl ether (180 parts by mass), a precipitated salt was separated by filtration, and diisopropyl ether was added to the filtrate, affording a diisopropyl ether solution of polycarbosilane (g-1). The concentration of the polycarbosilane (g-1) in the diisopropyl ether solution was 10% by mass. Polycarbosilane (g-1) had an Mw of 700.

### [Synthesis Examples 2 to 5] (Synthesis of Polycarbosilanes (g-2) to (g-5))

Diisopropyl ether solutions of polycarbosilanes (g-2) to (g-5) were obtained in the same manner as in Synthesis example 1 except that the monomers of the types and use amounts given in the following Table 1 were used, respectively. The Mw of the polycarbosilane (g) in the obtained solution of the polycarbosilane (g) and the concentration (% by mass) of the polycarbosilane (g) in the diisopropyl ether solution are shown together in Table 1. In Table 1, “-” indicates that the corresponding monomer was not used.

TABLE 1

| | Polycarbo- | Charged amount of each | | | | | | Solid | |
|---|---|---|---|---|---|---|---|---|---|
| | silane | monomer (mol %) | | | | | | concentration | |
| | (g) | H-1 | H-2 | H-3 | S-1 | S-2 | S-3 | (% by mass) | Mw |
| Synthesis | g-1 | 50 | — | — | — | 15 | 35 | 10 | 700 |
| Example 1 | | | | | | | | | |
| Synthesis | g-2 | 50 | — | — | 5 | 15 | 30 | 10 | 800 |
| Example 2 | | | | | | | | | |
| Synthesis | g-3 | 40 | — | 5 | 10 | 15 | 30 | 10 | 700 |
| Example 3 | | | | | | | | | |
| Synthesis | g-4 | — | 55 | — | 5 | — | 40 | 10 | 900 |
| Example 4 | | | | | | | | | |
| Synthesis | g-5 | — | — | 50 | 20 | — | 30 | 10 | 800 |
| Example 5 | | | | | | | | | |

### [Synthesis Example 6] (Synthesis of Polycarbosilane (D2-3-1))

In a reaction vessel, a diisopropyl ether solution of polycarbosilane (g-1) was dissolved in 90 parts by mass of methanol. A temperature in the reaction vessel was adjusted to 30° C., and 8 parts by mass of a 3.2% by mass aqueous solution of oxalic acid was added dropwise thereto over 20 minutes with stirring. The completion of the dropwise addition was taken as the start time of a reaction, and the reaction was performed at 40° C. for 4 hours. After the completion of the reaction, the inside of the reaction vessel was cooled to 30° C. or lower. To the reaction solution cooled was added 198 parts by mass of propylene glycol monoethyl ether acetate, and then water, alcohols produced through the reaction, and surplus propylene glycol monoethyl ether acetate were removed using an evaporator, affording a propylene glycol monomethyl ether acetate solution of polycarbosilane (D2-3-1). The concentration of polycarbosilane (D2-3-1) in the propylene glycol monomethyl ether acetate solution was 5% by mass. Polycarbosilane (D2-3-1) had an Mw of 2,500.

### [Synthesis Examples 7 to 10] (Synthesis of Polycarbosilanes (D2-3-2) to (D2-3-5))

Propylene glycol monomethyl ether acetate solutions of polycarbosilanes (D2-3-2) to (D2-3-5) were obtained in the same manner as in Synthesis example 6 except that polycarbosilanes (g-2) to (g-5) were used. The concentrations of polycarbosilanes (D2-3-2) to (D2-3-5) in the propylene glycol monomethyl ether acetate solutions were 5% by mass. Polycarbosilane (D2-3-2) had an Mw of 1,800, polycarbosilane (D2-3 3) had an Mw of 2,100, polycarbosilane (D2-3-4) had an Mw of 1,300, and polycarbosilane (D2-3-5) had an Mw of 1,800.

### (Polycarbosilane [D2-3])

D2-3-1: Polycarbosilane (D2-3-1) synthesized as described above (Mw: 2,500)
D2-3-2: Polycarbosilane (D2-3-2) synthesized as described above (Mw: 1,800)
D2-3-3: Polycarbosilane (D2-3-3) synthesized as described above (Mw: 2,100)
D2-3-4: Polycarbosilane (D2-3-4) synthesized as described above (Mw: 1,300)
D2-3-5: Polycarbosilane (D2-3-5) synthesized as described above (Mw: 1,800)

### (Additive [D4])

Compounds (D-1) to (D-3) as the crosslinking agent [D4-1] and compound (D-4) as the crosslinking accelerator [D4-2] are given below.
D-1: Compound represented by the following formula (d-1)
D-2: Compound represented by the following formula (d-2)
D-3: Compound represented by the following formula (d-3)
D-4: Compound represented by the following formula (d-4)

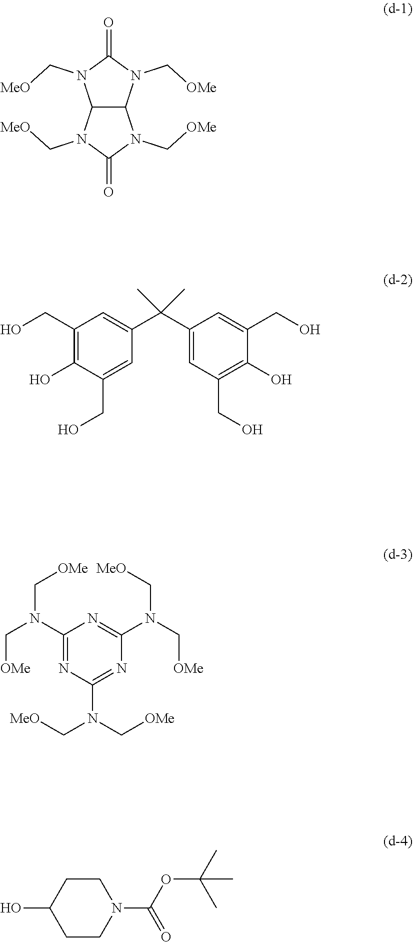 [SMILES: COCN1C(=O)N(COC)C2C1N(COC)C(=O)N2COC ; CC(C)(c1cc(CO)c(O)c(CO)c1)c1cc(CO)c(O)c(CO)c1 ; COCN(COC)c1nc(N(COC)COC)nc(N(COC)COC)n1 ; CC(C)(C)OC(=O)N1CCC(O)CC1]

### (Solvent [E])

Solvents (E-1) to (E-2) as the solvent [E] are given below.
E-1: Propylene glycol monomethyl ether acetate
E-2: Propylene glycol monoethyl ether

### Example 1

In 97.0 parts by mass of the solvent (E-1) were dissolved 0.3 parts by mass of the thermal acid generator (A-1) and 2.7 parts by mass of the organic polymer (D1-2). The resulting solution was filtered through a membrane filter having a pore size of 0.45 μm to prepare a composition for forming a resist underlayer film (J-1).

### Examples 2 to 43

Compositions for resist underlayer film formation (J-2) to (J-43) were prepared by operating in the same manner as in Example 1 except that the components with the types and the contents given in Table 2 were used. Note that “-” in the following Table 2 indicates that the corresponding component was not used.

TABLE 2

| | | | | | | Photo-base | | Organic | | | | | |
|---|---|---|---|---|---|---|---|---|---|---|---|---|---|
| | | | | Acid group- | | generator [C1] and | | polymer [D1] | | | | | |
| | Composition | Acid generating | | containing | | base-containing | | and inorganic | | | | | |
| | for forming | component [A] | | component [B] | | component [C2] | | polymer [D2] | | Additive [D4] | | Solvent [E] | |
| | a resist | | Content | | Content | | Content | | Content | | Content | | Content |
| | underlayer | | (parts by | | (parts by | | (parts by | | (parts by | | (parts by | | (parts by |
| | film | Type | mass) | Type | mass) | Type | mass) | Type | mass) | Type | mass) | Type | mass) |
| Example 1 | J-1 | A-1 | 0.3 | — | — | — | — | D1-2 | 2.7 | — | — | E-1 | 97.0 |
| Example 2 | J-2 | A-2 | 0.3 | — | — | — | — | D1-2 | 2.7 | — | — | E-1 | 97.0 |
| Example 3 | J-3 | A-3 | 0.3 | — | — | — | — | D1-2 | 2.7 | — | — | E-1 | 97.0 |
| Example 4 | J-4 | A-4 | 2.7 | — | — | — | — | — | — | — | — | E-1 | 97.0 |
| Example 5 | J-5 | A-5 | 0.3 | — | — | — | — | D1-2 | 2.7 | — | — | E-1 | 97.0 |
| Example 6 | J-6 | A-6 | 0.3 | — | — | — | — | D1-2 | 2.7 | — | — | E-1 | 97.0 |
| Example 7 | J-7 | — | — | B-1 | 2.7 | — | — | — | — | — | — | E-1 | 97.0 |
| Example 8 | J-8 | A-1 | 0.3 | — | — | — | — | D1-1 | 2.7 | — | — | E-1 | 97.0 |
| Example 9 | J-9 | A-1 | 0.3 | — | — | — | — | D1-3 | 2.7 | — | — | E-1 | 97.0 |
| Example 10 | J-10 | A-1 | 0.3 | — | — | — | — | D1-4 | 2.7 | — | — | E-1 | 97.0 |
| Example 11 | J-11 | A-1 | 0.3 | — | — | — | — | D1-5 | 2.7 | — | — | E-1 | 97.0 |
| Example 12 | J-12 | A-1 | 0.3 | — | — | — | — | D1-6 | 2.7 | — | — | E-1 | 97.0 |
| Example 13 | J-13 | A-1 | 0.3 | — | — | — | — | D2-1-1 | 2.7 | — | — | E-2 | 97.0 |
| Example 14 | J-14 | A-1 | 0.3 | — | — | — | — | D2-1-2 | 2.7 | — | — | E-2 | 97.0 |
| Example 15 | J-15 | A-1 | 0.3 | — | — | — | — | D2-1-3 | 2.7 | — | — | E-2 | 97.0 |
| Example 16 | J-16 | A-1 | 0.3 | — | — | — | — | D2-1-4 | 2.7 | — | — | E-2 | 97.0 |
| Example 17 | J-17 | A-1 | 0.3 | — | — | — | — | D2-2-1 | 2.7 | — | — | E-2 | 97.0 |
| Example 18 | J-18 | A-1 | 0.3 | — | — | — | — | D2-2-2 | 2.7 | — | — | E-2 | 97.0 |
| Example 19 | J-19 | — | — | — | — | C1-1 | 0.3 | D1-2 | 2.7 | — | — | E-1 | 97.0 |
| Example 20 | J-20 | — | — | — | — | C1-2 | 0.3 | D1-2 | 2.7 | — | — | E-1 | 97.0 |
| Example 21 | J-21 | — | — | — | — | C1-3 | 0.3 | D1-2 | 2.7 | — | — | E-1 | 97.0 |
| Example 22 | J-22 | — | — | — | — | C2-1 | 0.3 | D1-2 | 2.7 | — | — | E-1 | 97.0 |
| Example 23 | J-23 | — | — | — | — | C1-1 | 0.3 | D1-1 | 2.7 | — | — | E-1 | 97.0 |
| Example 24 | J-24 | — | — | — | — | C1-1 | 0.3 | D1-3 | 2.7 | — | — | E-1 | 97.0 |
| Example 25 | J-25 | — | — | — | — | C1-1 | 0.3 | D1-4 | 2.7 | — | — | E-1 | 97.0 |
| Example 26 | J-26 | — | — | — | — | C1-1 | 0.3 | D1-5 | 2.7 | — | — | E-1 | 97.0 |
| Example 27 | J-27 | — | — | — | — | C1-1 | 0.3 | D1-6 | 2.7 | — | — | E-1 | 97.0 |
| Example 28 | J-28 | — | — | — | — | C1-1 | 0.3 | D2-1-1 | 2.7 | — | — | E-2 | 97.0 |
| Example 29 | J-29 | — | — | — | — | C1-1 | 0.3 | D2-1-2 | 2.7 | — | — | E-2 | 97.0 |
| Example 30 | J-30 | — | — | — | — | C1-1 | 0.3 | D2-1-3 | 2.7 | — | — | E-2 | 97.0 |
| Example 31 | J-31 | — | — | — | — | C1-1 | 0.3 | D2-1-4 | 2.7 | — | — | E-2 | 97.0 |
| Example 32 | J-32 | — | — | — | — | C1-1 | 0.3 | D2-2-1 | 2.7 | — | — | E-2 | 97.0 |
| Example 33 | J-33 | — | — | — | — | C1-1 | 0.3 | D2-2-2 | 2.7 | — | — | E-2 | 97.0 |
| Example 34 | J-34 | A-1 | 0.3 | — | — | — | — | D1-2 | 2.5 | D-1 | 0.2 | E-1 | 97.0 |
| Example 35 | J-35 | A-1 | 0.3 | — | — | — | — | D1-2 | 2.5 | D-1 | 0.2 | E-1 | 97.0 |
| Example 36 | J-36 | A-2 | 0.3 | — | — | — | — | D1-2 | 2.5 | D-2 | 0.2 | E-1 | 97.0 |
| Example 37 | J-37 | A-3 | 0.3 | — | — | — | — | D1-2 | 2.5 | D-3 | 0.2 | E-1 | 97.0 |
| Example 38 | J-38 | A-5 | 0.3 | — | — | — | — | D1-2 | 2.5 | D-4 | 0.2 | E-1 | 97.0 |
| Example 39 | J-39 | A-1 | 0.3 | — | — | — | — | D2-3-1 | 2.7 | — | — | E-1 | 97.0 |
| Example 40 | J-40 | A-1 | 0.3 | — | — | — | — | D2-3-2 | 2.7 | — | — | E-1 | 97.0 |
| Example 41 | J-41 | A-1 | 0.3 | — | — | — | — | D2-3-3 | 2.7 | — | — | E-1 | 97.0 |
| Example 42 | J-42 | A-1 | 0.3 | — | — | — | — | D2-3-4 | 2.7 | — | — | E-1 | 97.0 |
| Example 43 | J-43 | A-1 | 0.3 | — | — | — | — | D2-3-5 | 2.7 | — | — | E-1 | 97.0 |

### <Preparation of Substrate>

### [Preparation of Substrate (S-1)]

A substrate (S-1) including a 12-inch silicon wafer and a silicon dioxide film having a thickness of 20 nm formed thereon was prepared.

### [Production of Substrate (S-2)]

A substrate (S-2) including a 12-inch silicon wafer and a silicon carbide film having a thickness of 20 nm formed thereon was prepared.

### [Preparation of Substrate (S-3)]

The composition for forming a resist underlayer film prepared as described above was applied to the substrate (S-1) by spin-coating using a spin-coater (“CLEAN TRACK ACT12”, available from Tokyo Electron Limited), and thereafter heating was conducted at 250° C. for 60 sec to form a resist underlayer film having an average thickness of 5 nm and prepare a substrate (S-3).

### <Formation of Metal-Containing Resist Film>

On a surface of the substrate (S-1), substrate (S-2) or substrate (S-3) prepared as described above, a metal-containing resist film having a thickness of 5 nm was formed using a CVD device, at 350° C., at a methyltin trichloride flow rate of 200 ccm, and at a CO₂ flow rate of 1000 sccm.

### <Formation of Resist Pattern>

The metal-containing resist film prepared as described above was irradiated with extreme ultraviolet rays using an EUV scanner (“TWINSCAN NXE:3300B”, available from ASML Co. (NA=0.3; Sigma=0.9; quadrupole illumination, with a 1:1 line and space mask having a line width of 16 nm in terms of a dimension on wafer)). Thereafter, development was performed by a paddle method for 60 seconds using ethanol/water (volume ratio: 70/30) heated to 40° C., and drying was then performed, affording a substrate for evaluation on which a resist pattern had been formed.

### <Evaluation>

Pattern rectangularity was evaluated in accordance with the following method. The evaluation results are given in the following Table 3. In Table 3, “-” indicates that no composition for forming a resist underlayer film was applied.

### [Pattern Rectangularity]

A scanning electron microscope (“SU8220” available from Hitachi High-Technologies Corporation) was used for length measurement and observation of the resist pattern of the substrate for evaluation. The pattern rectangularity was evaluated as “A” (good) when the cross-sectional shape of the pattern was rectangular, “B1” (poor) when trailing was present in the cross section of the pattern, and “B2” (poor) when collapse of the resist pattern was present.

TABLE 3

| | | Composition | |
|---|---|---|---|
| | | for forming | |
| | | a resist | Pattern |
| | Substrate | underlayer film | rectangularity |
| Example 1 | S-3 | J-1 | A |
| Example 2 | S-3 | J-2 | A |
| Example 3 | S-3 | J-3 | A |
| Example 4 | S-3 | J-4 | A |
| Example 5 | S-3 | J-5 | A |
| Example 6 | S-3 | J-6 | A |
| Example 7 | S-3 | J-7 | A |
| Example 8 | S-3 | J-8 | A |
| Example 9 | S-3 | J-9 | A |
| Example 10 | S-3 | J-10 | A |
| Example 11 | S-3 | J-11 | A |
| Example 12 | S-3 | J-12 | A |
| Example 13 | S-3 | J-13 | A |
| Example 14 | S-3 | J-14 | A |
| Example 15 | S-3 | J-15 | A |
| Example 16 | S-3 | J-16 | A |
| Example 17 | S-3 | J-17 | A |
| Example 18 | S-3 | J-18 | A |
| Example 19 | S-3 | J-19 | A |
| Example 20 | S-3 | J-20 | A |
| Example 21 | S-3 | J-21 | A |
| Example 22 | S-3 | J-22 | A |
| Example 23 | S-3 | J-23 | A |
| Example 24 | S-3 | J-24 | A |
| Example 25 | S-3 | J-25 | A |
| Example 26 | S-3 | J-26 | A |
| Example 27 | S-3 | J-27 | A |
| Example 28 | S-3 | J-28 | A |
| Example 29 | S-3 | J-29 | A |
| Example 30 | S-3 | J-30 | A |
| Example 31 | S-3 | J-31 | A |
| Example 32 | S-3 | J-32 | A |
| Example 33 | S-3 | J-33 | A |
| Example 34 | S-3 | J-34 | A |
| Example 35 | S-3 | J-35 | A |
| Example 36 | S-3 | J-36 | A |
| Example 37 | S-3 | J-37 | A |
| Example 38 | S-3 | J-38 | A |
| Example 39 | S-3 | J-39 | A |
| Example 40 | S-3 | J-40 | A |
| Example 41 | S-3 | J-41 | A |
| Example 42 | S-3 | J-42 | A |
| Example 43 | S-3 | J-43 | A |
| Comparative | S-1 | — | B1 |
| Example 1 | | | |
| Comparative | S-2 | — | B2 |
| Example 2 | | | |

As can be seen from the results in Table 3, Examples, in which a resist underlayer film was formed, were superior in pattern rectangularity to Comparative Examples, in which no resist underlayer film was formed.

According to the method for manufacturing a semiconductor substrate of the present disclosure, a composition for forming a resist underlayer film superior in pattern rectangularity is used, so that a semiconductor substrate having a good pattern shape can be efficiently manufactured. Therefore, the method for manufacturing a semiconductor substrate can suitably be used for, for example, manufacturing semiconductor devices expected to be further microfabricated in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A method for manufacturing a semiconductor substrate, the method comprising:
forming a resist underlayer film directly or indirectly on a substrate by applying a composition for forming a resist underlayer film;
forming a metal-containing resist film on the resist underlayer film;
exposing the metal-containing resist film; and
dissolving an exposed portion of the exposed metal-containing resist film with a developer to form a resist pattern.

2. The method according to claim 1, wherein the metal-containing resist film is formed by performing a deposition of a metal compound.

3. The method according to claim 2, wherein the deposition is performed by CVD or ALD.

4. The method according to claim 2, wherein the metal compound comprises at least one compound selected from the group consisting of a haloalkyl Sn, an alkoxyalkyl Sn, and an amidoalkyl Sn.

5. The method according to claim 2, wherein the metal compound comprises at least one compound selected from the group consisting of trimethyltin chloride, dimethyltin dichloride, methyltin trichloride, tris(dimethylamino)methyltin(IV), and (dimethylamino)trimethyltin(IV).

6. The method according to claim 1, wherein the metal-containing resist film comprises an organotin oxide.

7. The method according to claim 1, wherein the metal-containing resist film is exposed with an extreme ultraviolet ray.

8. The method according to claim 1, wherein the developer comprises water, an alcohol, or a combination thereof.

9. The method according to claim 1, wherein a temperature of the developer is 40° C. or higher at least when the exposed portion is dissolved.

10. The method for according to claim 1, wherein the composition for forming a resist underlayer film comprises:
at least one component selected from the group consisting of an acid generating component, an acid group-containing component, a photo-base generator, and a base-containing component; and
a solvent.

11. A composition comprising:
at least one component selected from the group consisting of an acid generating component, an acid group-containing component, a photo-base generator, and a base-containing component; and
a solvent,
wherein the composition is suitable for forming a resist underlayer film in a method for manufacturing a semiconductor substrate, the method comprising:
forming a resist underlayer film directly or indirectly on a substrate by applying the composition;
forming a metal-containing resist film on the resist underlayer film;
exposing the metal-containing resist film; and
dissolving an exposed portion of the exposed metal-containing resist film with a developer to form a resist pattern.
